(19) United States
(12) Patent Application Publication
(10) Pub. No.: US 2021/0220288 A1
(43) Pub. Date: Jul. 22, 2021

(54) NATURAL NANOPARTICLE-MEDICINE COMPOSITION FOR TREATING ALZHEIMER'S DISEASE, PREPARATION METHOD THEREFOR AND APPLICATION THEREOF

(71) Applicant: CHINA PHARMACEUTICAL UNIVERSITY, Nanjing (CN)

(72) Inventors: Jianping ZHOU, Nanjing (CN); Yang DING, Nanjing (CN); Huaqing ZHANG, Nanjing (CN)

(21) Appl. No.: 16/755,306

(22) PCT Filed: Nov. 29, 2018
(86) PCT No.: PCT/CN2018/118109

§ 371 Date: Apr. 10, 2020

(30) Foreign Application Priority Data
Jul. 30, 2018 (CN) 201810855065.2

(51) Int. Cl. A61K 9/51; A61K 31/445; A61K 31/12; A61K 31/4748; A61K 31/7105; A61K 38/08; A61K 31/13; A61K 31/5415

(52) U.S. Cl. CPC A61K 9/5123; A61K 9/5169; A61K 31/445; A61K 31/12; B82Y 5/00; A61K 31/7105; A61K 38/08; A61K 31/13; A61K 31/5415; A61K 31/4748

(57) ABSTRACT

The present invention belongs to the field of medicinal preparations, and more particularly relates to preparation and application of a natural nanoparticle-drug composition for Alzheimer's disease (AD) therapy. A technical problem solved by the present invention is to construct the natural nanoparticle-drug composition by extracting natural lipoprotein nanoparticles and recombining AD treatment drugs to achieve the purpose of anti-AD combination treatment. The present invention provides a prescription composition, a preparation process, property evaluation and application of the natural nanoparticle-drug composition, the natural nanoparticle-drug composition is administered in modes of intravenous injection, oral administration, nasal administration and the like, new ideas and new technologies are provided for research and development of new AD drugs, and important research value and clinical research prospects are achieved.

### TECHNICAL FIELD

The present invention belongs to the field of medicinal preparations, and more particularly relates to preparation and application of a natural nanoparticle-drug composition for Alzheimer's disease therapy.

### BACKGROUND ART

Alzheimer's disease (AD), also known as senile dementia, is a central nervous system degenerative disease mainly characterized by progressive cognitive disorder and memory loss. With the development of a global aging process, the incidence of AD continues to increase and AD seriously affects human health and quality of life. World Alzheimer Report indicates that the number of patients with dementia is expected to increase nearly twice every 20 years, and that the number of patients is about to break through 100 million by 2050. Therefore, Alzheimer's disease has become a major public health issue and an increasingly prominent social problem of common concern of all countries in the world.

However, there is still a lack of effective treatment means for AD, and the main treatment bottleneck is the lack of treatment means effectively designed for disease processes and pathological reasons and the difficulty of brain drug delivery caused by blood brain barrier (BBB). Currently, drugs for clinical AD treatment are only configured to relieve disease symptoms, and five kinds of drugs which are widely applied clinically are four kinds of cholinesterase inhibitors (donepezil, tacrine, galantamine and rivastigmine) and one kind of glutamate NMDA receptor antagonist (memantine). The cholinesterase inhibitors are combined with a target enzyme to form a covalent complex, so that the target enzyme temporarily loses activity, the degradation of released acetylcholine by cholinergic neurons is delayed, the effects of acetylcholine in the cerebral cortex, hippocampus and other parts are selectively enhanced, the activity of cholinergic nerves is promoted, and the decline of learning and memory capacity caused by cholinergic deficiency is relieved. The NMDA receptor antagonist acts on a glutamate neurotransmitter system in the brain to replace magnesium ions to occupy NMDA access, inhibit calcium ion influx and improve cognition. The above five drugs can only relieve symptoms to a certain extent and cannot effectively inhibit the disease process, so the design and development of a disease treatment means aiming at the pathological features of AD are very necessary. In another major bottleneck of AD treatment, the blood brain barrier is a double barrier formed by brain capillary endothelial cells, neuroglia cells and choroid plexus, and can prevent 98% of drugs from penetrating into brain tissues, which is also the problem of gastrointestinal adverse reactions caused by non-specific distribution of clinical AD treatment drugs at present. Therefore, from an AD pathological mechanism, it is very important to design therapeutic methods to reverse the disease and jointly develop BBB high-permeability drug carriers for AD treatment.

Senile plaques (SP) formed by deposition of neuronal extracellular β amyloid protein (Aβ) and neurofibrillary tangles (NFT) formed by aggregation of neuronal intracellular hyperphosphorylated Tau protein are major pathological features of AD. Under normal conditions, the production and clearance (extracellular degradation, microglia-mediated phagocytosis, and transport clearance) of Aβ in the brain are in a state of dynamic equilibrium, and when the equilibrium is disrupted, Aβ is overproduced and abnormally deposited in the brain, thereby causing synaptic dysfunction of peripheral neurons, Tau protein hyperphosphorylation, oxidative stress and secondary inflammatory reactions, which leads to neuronal degeneration and death and ultimately produces dementia. Therefore, Aβ is the most important disease biomarker and therapeutic target of AD, and how to reduce the level of Aβ₁₋₄₂ in the brain is also an important strategy of AD treatment Immunotherapy to eliminate brain Aβ deposition and improve the pathological process of AD is the most effective treatment means in preclinical study of AD drugs. However, immunotherapy has failed in preclinical studies due to the problems of poor brain targeting ability, misrecognition of neuronal cell APP fragments, immune side effects and the like. Therefore, there is an urgent need to develop new therapeutic strategies that can overcome the BBB, specifically accumulate in the focal region of the brain, recognize neurotoxicity Aβ accumulated extracellularly in neurons, and effectively eliminate Aβ without obvious side effects. Song et al. constructed recombinant high-density lipoprotein apoE3-rHDL nanoparticles by using in vitro phospholipid and apolipoprotein E3 recombination, which could clear brain Aβ across the BBB after intravenous injection to treat AD. Yao et al. designed α-mangostin-encapsulated PEG-PLA nanoparticles to promote brain Aβ clearance by up-regulating an expression of low-density lipoprotein receptors so as to achieve the treatment effect of AD. Although nanoparticles designed for the Aβ elimination strategy have been reported in literatures, the technology of how to design and prepare nano-drugs with high brain targeting ability, high Aβ affinity and targeted elimination ability, realize drug-carrier integration and improve the effect of multi-mode treatment of AD needs to be developed.

Lipoprotein is an endogenous micro-particle playing an important role in lipid metabolism in plasma and consists of phospholipid, cholesterol, apolipoprotein and non-polar lipid. Recent studies have shown that the expression level of the lipoprotein in the brain plays an important role in Aβ clearance, and intracerebral Aβ deposition can be cleared by tail vein injection of apolipoprotein to improve learning and memory capacity of AD model mice. However, the source of natural lipoprotein is scarce, the preparation is complicated and the quality controllability is not strong, so recombinant lipoprotein as the in vitro-recombinant form of endogenous lipoprotein has attracted much attention because of its unique “bionic” characteristics. The recombinant lipoprotein possesses the unique amphipathic (external hydrophilic-internal hydrophobic) structure of the natural lipoprotein, achieves various loading forms (surface loading, covalent modification, inner core embedding and the like), and is beneficial to drug loading and targeted modification. Meanwhile, the recombinant lipoprotein can be mediated by a scavenger receptor (SR-BI receptor) highly expressed by BBB endothelial cells and a low-density lipoprotein related receptor (LDLR-1) to cross the BBB, so that high-efficiency brain targeted drug delivery is realized, and the recombinant lipoprotein is a good brain targeted delivery carrier. Based on the high-efficiency Aβ clearance capacity of the recombinant lipoprotein and the brain targeted delivery potential, through reasonable design of a combined treatment mode, the AD symptoms can be effectively relieved, the disease process can be inhibited, and even AD can be reversed. A preparation method of the recombinant lipoprotein has been reported in literatures and patents, but there are some problems, such as limited sources of raw materials, complex preparation processes and failure to simulate the endogenous lipoprotein. CN104324007A discloses a preparation technology and application of natural lipid nanoparticles, in particular to efficient tumor targeting nanoparticles prepared by extracting apolipoprotein and phospholipid from a plasma component IV and recombining them with antitumor drugs in vitro. The method is suitable for large-scale production, but the source of raw materials of the method is limited, the purity of the extracted lipoprotein is not high, and meanwhile, there is a need for improvement in delivery of antitumor drugs, extraction of high-purity lipoprotein, expansion of raw material sources and achievement of anti-AD application. CN104138595A discloses preparation of bionic recombinant high-density lipoprotein and application of the bionic recombinant high-density lipoprotein in prevention and treatment of AD, recombinant high-density lipoprotein is constructed through in vitro lipid and apolipoprotein recombination, targeted Aβ clearance is realized to treat AD, but the lipoprotein composition involved in the method is only high-density lipoprotein, and the composition is not completely the same as that of natural lipoprotein, so that the physiological function of the natural lipoprotein cannot be well reproduced, and meanwhile, the recombinant high-density lipoprotein is not used as a brain targeting drug carrier to solve the problem of AD brain targeted drug delivery. Therefore, it has not been reported how to prepare recombinant lipoprotein which highly restores the physiological function of the endogenous lipoprotein, efficiently carry drugs for achieving brain targeted delivery and the like.

### SUMMARY OF THE INVENTION

A technical problem to be solved by the present invention is to provide a preparation process of a natural nanoparticle-drug composition for Alzheimer's disease therapy and application of the natural nanoparticle-drug composition for Alzheimer's disease therapy.

In order to solve the above problem, the present invention provides a prescription composition and the preparation process of the natural nanoparticle-drug composition and application of the natural nanoparticle-drug composition in Alzheimer's disease therapy.

Preferably, the natural nanoparticle-drug composition has brain targeting ability and amyloid protein targeting ability, and by a total mass of a prescription, content of natural nanoparticles accounts for 50-99% of the total mass of the prescription and content of drugs accounts for 1-50% of the total mass of the prescription.

Preferably, the natural nanoparticles are natural nanoparticles extracted from plasma components or expressed through genetic engineering and having an anti-Alzheimer's effect, and a particle size range of the natural nanoparticles is 20-200 nm.

Preferably, the natural nanoparticles are natural lipoprotein nanoparticles, and lipoprotein is essentially composed of apolipoprotein and lipid and is selected from one or more of high-density lipoprotein, low-density lipoprotein and very-low-density lipoprotein.

Preferably, the apolipoprotein is extracted from plasma or obtained through genetic engineering and is selected from one or more of apolipoprotein apoA-I, apoA-II, apoE, apoC, apoB and apolipoprotein mimetic peptide.

Preferably, the lipid is selected from one or more of cholesterol, cholesterol ester, phosphatidylcholine, triglyceride, ceramide, ganglioside and cephalin.

Preferably, Alzheimer's disease treatment drugs are selected from any one or derivatives of cholinesterase inhibitors, aspartic acid receptor antagonists, antioxidants, anti-inflammatory drugs, protein drugs, polypeptide drugs and gene drugs, and selected from one or more of donepezil, huperzine A, memantine, curcumin, methylene blue, NAP polypeptide and siRNA.

Preferably, the preparation process of the natural nanoparticle-drug composition is an ultrasonic drug loading method (see Embodiment 1 for details), and includes the following specific steps:

(1) dispersing natural lipoprotein nanoparticles extracted from plasma or through genetic engineering into distilled water to form a natural nanoparticle suspension;

(2) dissolving drugs of a prescription dosage in a drug solvent, dropwise adding the natural nanoparticle suspension in step (1), and carrying out probe ultrasonic dispersion under an ice bath for 15-60 min; and

(3) after ultrasonic treatment is finished, when the drug solvent is an organic solvent, carrying out reduced pressure rotary evaporation for 5-20 min at 20-40° C. to remove the drug solvent; when the drug solvent is water, not carrying out rotary evaporation for removing the solvent; and then carrying out filtration through a 0.22 μm filter membrane, and freeze-drying to obtain the natural nanoparticle-drug composition.

Preferably, when the drug is a water-soluble drug, the drug solvent is distilled water; and when the drug is a fat-soluble drug, the drug solvent is ethanol.

Preferably, the preparation process of the natural nanoparticle-drug composition is a recombination ultrasonic method (see Embodiment 1 for details), and includes the following specific steps:

(1) weighing lipid of a prescription dosage, and adding the lipid into a membrane-forming solvent to form a lipid solution;

(2) weighing apolipoprotein of a prescription dosage, and dissolving the apolipoprotein in distilled water to form an apolipoprotein solution;

(3) weighing drugs of a prescription dosage, and dissolving the drugs in the above corresponding components according to the principle of “similarity compatibility”: dissolving fat-soluble drugs in the lipid solution and water-soluble drugs in the apolipoprotein solution;

(4) carrying out rotary evaporation on the lipid solution at 20-40° C. for 0.5-2 h to form a membrane, and carrying out overnight vacuum drying;

(5) adding the distilled water into the lipid membrane in step (4) and carrying out rotary evaporation at 20-40° C. for 5-20 min for hydration;

(6) after the hydration is finished, carrying out probe ultrasonic dispersion under an ice bath for 5-20 min to obtain drug-loaded nanoparticles; and

(7) adding the apolipoprotein solution into the drug-loaded nanoparticles in step (6), and magnetically stirring at room temperature for incubation for 24-48 h; and after the incubation is finished, carrying out filtration through a 0.22 μm filter membrane, and freeze-drying to obtain the natural nanoparticle-drug composition.

Preferably, the natural nanoparticle-drug composition is administered after being diluted with a buffer solution, the buffer solution can be normal saline, a phosphate buffer solution, a 5% glucose solution, an HEPES buffer solution or a Tris buffer solution, a drug administration mode is intravenous injection, or oral administration, or nasal administration, and the natural nanoparticle-drug composition is configured to treat Alzheimer's disease.

The present invention is characterized in:

The natural nanoparticle-drug composition is constructed by extracting the natural lipoprotein nanoparticles and encapsulating anti-AD drugs in an in-vitro recombination mode. The composition has high bionicity and biological safety, can restore the high blood-brain barrier permeability of endogenous lipoprotein, completes the targeted clearance of the focal region Aβ while realizing the brain delivery of AD treatment drugs, realizes a multi-mode AD treatment effect, can solve the problems of difficult AD treatment drug delivery, single treatment mode and the like at present, and has wide application prospects.

According to the present invention, the natural nanoparticle-drug composition for Alzheimer's disease therapy is constructed through in-vitro recombination of the natural lipoprotein and anti-Alzheimer's chemical drugs, so that brain targeted delivery of the AD treatment drugs can be realized, and meanwhile, an Aβ clearance mechanism of body natural lipoprotein is simulated to realize multi-mode combined treatment, which has an important regulation effect on an AD process.

The natural nanoparticle-drug composition for Alzheimer's disease therapy is constructed through the in-vitro recombination of the natural lipoprotein and the anti-Alzheimer's drugs, so that brain targeted delivery of the AD treatment drugs can be realized, difficulty in brain delivery of the drugs is solved, and multi-mode AD treatment is realized by combining with lipoprotein intracerebral Aβ targeted clearance. The present invention has the following advantages:

(1) High homology and biocompatibility of carriers: the composition of the adopted lipoprotein is completely the same as that of the natural lipoprotein, so that the biological safety and the biological functional integrity of the lipoprotein are guaranteed, and in particular, the brain targeting ability of endogenous lipoprotein and the ability of intracerebral Aβ targeted clearance are guaranteed.

(2) High BBB permeability and AD active targeting ability: by utilizing the high BBB permeability of the lipoprotein and the Aβ amyloid plaque targeting ability of the apolipoprotein, effective positioning and release of the encapsulated drugs in AD lesion regions can be realized, and the problem of intracerebral targeted delivery of the drugs is solved.

(3) Combination treatment: aiming at a formation and development mechanism of AD, the anti-AD drugs are selected to alleviate symptoms and combined with the Aβ targeted clearance function of the lipoprotein to achieve the high-efficiency combined treatment of an AD lesion nervous system.

(4) Simple preparation process and high drug loading capacity: the natural nanoparticle-drug composition with high drug content can conveniently prepared by carrying out in-vitro recombination on the natural lipoprotein and the anti-AD drugs through the ultrasonic drug loading method or the recombination ultrasonic method, and the simple preparation process lays a foundation for later process amplification.

The present invention provides the preparation process and application of the natural nanoparticle-drug composition for Alzheimer's disease therapy the physiological function of the natural lipoprotein is highly restored, high-efficiency brain targeted drug delivery and the targeted clearance of Aβ are achieved, and the purpose of the combination treatment of AD is achieved.

Compared with the prior art, the present invention has the following advantages:

(1) the natural nanoparticles adopted by the present invention are the natural lipoprotein nanoparticles, the physiological function of the endogenous lipoprotein is highly restored, and the high-efficiency brain targeting ability, the high Aβ affinity and the targeted clearance capacity are guaranteed;

(2) the present invention realizes high-efficiency drug encapsulation on the basis of ensuring the physiological activity of the natural nanoparticles, and the natural nanoparticle-drug composition is constructed, so that the problem of difficulty in brain delivery of the AD treatment drugs is solved;

(3) the natural nanoparticles are extracted from the plasma or expressed through genetic engineering, are wide in source, high in safety and easy to produce on a large scale, and have great clinical application potential; and

(4) in view of the preparation and the anti-AD application of the natural nanoparticle-drug composition for Alzheimer's disease therapy provided by the present invention, the anti-AD drugs are efficiently delivered to the brain with AD lesions, the purpose of the multi-mode combined treatment of the high-efficiency targeted clearance of Aβ is achieved, and a new idea and a new technology are provided for AD treatment.

### BRIEF DESCRIPTION OF THE DRAWINGS

FIG. 1 is a preparation and structural diagram of a natural nanoparticle-drug composition for resisting Alzheimer's disease.

FIG. 2 is a projection electron micrograph of the natural nanoparticle-drug composition for resisting Alzheimer's disease.

FIG. 3 is an in-vitro release diagram of the natural nanoparticle-drug composition for resisting Alzheimer's disease.

FIG. 4 is a cellular uptake inspection diagram of the natural nanoparticle-drug composition for resisting Alzheimer's disease.

FIG. 5 is an inspection diagram of the natural nanoparticle-drug composition for resisting Alzheimer's disease to promote Aβ uptake of microglia cells.

### DETAILED DESCRIPTION OF THE INVENTION

For the purpose of making objects, technical solutions and advantages of the present invention clearer, further detailed description will be made to the present invention in conjunction with specific embodiments. Obviously, the described embodiments are merely used to explain the present invention and are not intended to limit the scope of the present invention.

### Embodiment 1: A Preparation Process of a Natural Nanoparticle-Drug Composition for Resisting Alzheimer's Disease

1.1 An ultrasonic drug loading method for preparing a donepezil-loaded natural nanoparticle-drug composition

| | |
|---|---|
| Prescription composition: high-density lipoprotein | 10 mg |
| donepezil | 5 mg |
| ethanol | 1 mL |
| ultrapure water | 3 mL |

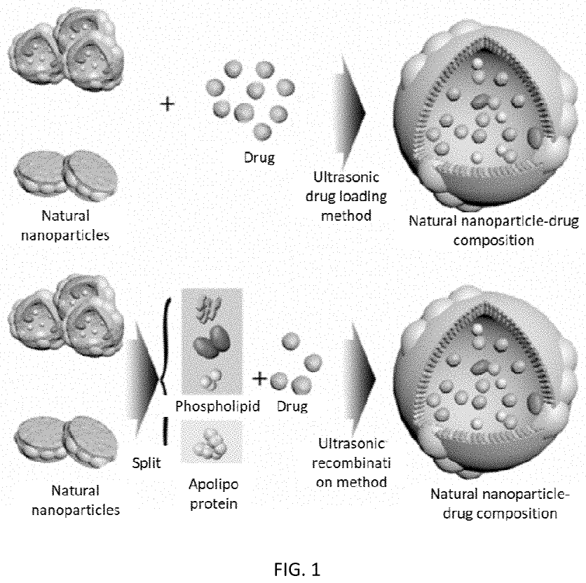

(1) As shown in FIG. 1, the donepezil-loaded natural nanoparticle-drug composition is prepared according to the ultrasonic drug loading method. 10 mg of the natural nanoparticles-the high-density lipoprotein of a prescription dosage are weighed and dispersed into the ultrapure water to form a natural high-density lipoprotein nanoparticle suspension.

(2) 5 mg of the donepezil of a prescription dosage is weighed and dissolved in ethanol. The natural nanoparticle suspension in step (1) is dropwise added. Probe ultrasonic dispersion is carried out under an ice bath for 60 min.

(3) After ultrasonic treatment is finished, reduced pressure rotary evaporation is carried out for 20 min at 37° C. to remove the drug solvent-ethanol. Then a 0.22 μm filter membrane is utilized for filtration. Freeze-drying is carried out to obtain the donepezil-loaded natural nanoparticle-drug composition.

1.2 An ultrasonic drug loading method for preparing a curcumin-loaded natural nanoparticle-drug composition

| | |
|---|---|
| Prescription composition: high-density lipoprotein | 5 mg |
| low-density lipoprotein | 4 mg |
| very-low-density lipoprotein | 1 mg |
| curcumin | 10 mg |
| ethanol | 1 mL |
| ultrapure water | 3 mL |

(1) As shown in FIG. 1, the curcumin-loaded natural nanoparticle-drug composition is prepared according to the ultrasonic drug loading method. The natural nanoparticles-the high-density lipoprotein, the low-density lipoprotein and the very-low-density lipoprotein of a prescription dosage are weighed and dispersed into the ultrapure water to form a natural high-density lipoprotein, low-density lipoprotein and very-low-density lipoprotein nanoparticle suspension.

(2) 10 mg of the curcumin of a prescription dosage is weighed and dissolved in ethanol. The natural nanoparticle suspension in step (1) is dropwise added. Probe ultrasonic dispersion is carried out under an ice bath for 15 min.

(3) After ultrasonic treatment is finished, reduced pressure rotary evaporation is carried out for 20 min at 37° C. to remove the drug solvent-ethanol. Then a 0.22 μm filter membrane is utilized for filtration. Freeze-drying is carried out to obtain the curcumin-loaded natural nanoparticle-drug composition.

1.3 An ultrasonic drug loading method for preparing a huperzine A-loaded natural nanoparticle-drug composition

| | |
|---|---|
| Prescription composition: low-density lipoprotein | 10 mg |
| huperzine A | 3 mg |
| ethanol | 1 mL |
| ultrapure water | 3 mL |

(1) As shown in FIG. 1, the huperzine A-loaded natural nanoparticle-drug composition is prepared according to the ultrasonic drug loading method. The natural nanoparticles-the low-density lipoprotein of a prescription dosage are weighed and dispersed into the ultrapure water to form a natural low-density lipoprotein nanoparticle suspension.

(2) 3 mg of the huperzine A of a prescription dosage is weighed and dissolved in ethanol. The natural nanoparticle suspension in step (1) is dropwise added. Probe ultrasonic dispersion is carried out under an ice bath for 25 min.

(3) After ultrasonic treatment is finished, reduced pressure rotary evaporation is carried out for 20 min at 37° C. to remove the drug solvent-ethanol. Then a 0.22 μm filter membrane is utilized for filtration. Freeze-drying is carried out to obtain the huperzine A-loaded natural nanoparticle-drug composition.

1.4 An ultrasonic drug loading method for preparing an siRNA-loaded natural nanoparticle-drug composition

| | |
|---|---|
| Prescription composition: high-density lipoprotein | 10 mg |
| Chol-siRNA | 50 μg |
| ethanol | 1 mL |
| ultrapure water | 3 mL |

(1) As shown in FIG. 1, the siRNA-loaded natural nanoparticle-drug composition is prepared according to the ultrasonic drug loading method. 10 mg of the natural nanoparticles-the high-density lipoprotein of a prescription dosage are weighed and dispersed into the ultrapure water to form a natural high-density lipoprotein nanoparticle suspension.

(2) 50 μg of the Chol-siRNA modified by cholesterol is weighed and dissolved in a PBS buffer solution. The natural nanoparticle suspension in step (1) is dropwise added. Probe ultrasonic dispersion is carried out under an ice bath for 15 min.

(3) After ultrasonic treatment is finished, a 0.22 μm filter membrane is utilized for filtration. Freeze-drying is carried out to obtain the siRNA-loaded natural nanoparticle-drug composition.

1.5 An ultrasonic drug loading method for preparing an NAP polypeptide-loaded natural nanoparticle-drug composition

| | |
|---|---|
| Prescription composition: high-density lipoprotein | 8 mg |
| low-density lipoprotein | 2 mg |
| NAP | 80 μg |
| ethanol | 1 mL |
| ultrapure water | 3 mL |

(1) As shown in FIG. 1, the NAP polypeptide-loaded natural nanoparticle-drug composition is prepared according to the ultrasonic drug loading method. 8 mg of the natural nanoparticles-the high-density lipoprotein and 2 mg of the natural nanoparticles-the low-density lipoprotein of a prescription dosage are weighed and dispersed into the ultrapure water to form a natural high-density lipoprotein and low-density lipoprotein nanoparticle suspension.

(2) 80 μg of the NAP polypeptide of a prescription dosage is weighed and dissolved in a Tris buffer solution. The natural nanoparticle suspension in step (1) is dropwise added. Probe ultrasonic dispersion is carried out under an ice bath for 15 min.

(3) After ultrasonic treatment is finished, a 0.22 μm filter membrane is utilized for filtration. Freeze-drying is carried out to obtain the NAP polypeptide-loaded natural nanoparticle-drug composition.

1.6 An ultrasonic drug loading method for preparing a memantine-loaded natural nanoparticle-drug composition

| | |
|---|---|
| Prescription composition: low-density lipoprotein | 5 mg |
| very-low-density lipoprotein | 10 mg |
| memantine | 8 mg |
| ethanol | 1 mL |
| ultrapure water | 3 mL |

(1) As shown in FIG. 1, the memantine-loaded natural nanoparticle-drug composition is prepared according to the ultrasonic drug loading method. 5 mg of the natural nanoparticles-the low-density lipoprotein and 10 mg of the natural nanoparticles-the very-low-density lipoprotein of a prescription dosage are weighed and dispersed into the ultrapure water to form a natural low-density lipoprotein and very-low-density lipoprotein nanoparticle suspension.

(2) 8 mg of the memantine of a prescription dosage is weighed and dissolved in ethanol. The natural nanoparticle suspension in step (1) is dropwise added. Probe ultrasonic dispersion is carried out under an ice bath for 10 min.

(3) After ultrasonic treatment is finished, reduced pressure rotary evaporation is carried out for 20 min at 37° C. to remove the drug solvent-ethanol. Then a 0.22 μm filter membrane is utilized for filtration. Freeze-drying is carried out to obtain the memantine-loaded natural nanoparticle-drug composition.

1.7 An ultrasonic drug loading method for preparing a methylene blue-loaded natural nanoparticle-drug composition

| | |
|---|---|
| Prescription composition: high-density lipoprotein | 10 mg |
| methylene blue | 6 mg |
| ethanol | 1 mL |
| ultrapure water | 3 mL |

(1) As shown in FIG. 1, the methylene blue-loaded natural nanoparticle-drug composition is prepared according to the ultrasonic drug loading method. 10 mg of the natural nanoparticles-the high-density lipoprotein of a prescription dosage are weighed and dispersed into the ultrapure water to form a natural high-density lipoprotein nanoparticle suspension.

(2) 6 mg of the methylene blue of a prescription dosage is weighed and dissolved in ethanol. The natural nanoparticle suspension in step (1) is dropwise added. Probe ultrasonic dispersion is carried out under an ice bath for 25 min.

(3) After ultrasonic treatment is finished, reduced pressure rotary evaporation is carried out for 20 min at 37° C. to remove the drug solvent-ethanol. Then a 0.22 μm filter membrane is utilized for filtration. Freeze-drying is carried out to obtain the methylene blue-loaded natural nanoparticle-drug composition.

1.8 A recombination ultrasonic method for preparing a donepezil-loaded natural nanoparticle-drug composition

| | |
|---|---|
| Prescription composition: apoA-I | 8 mg |
| apoE | 2 mg |
| phosphatidylcholine | 5 mg |
| cholesterol | 1 mg |
| donepezil | 5 mg |
| hydration medium | ultrapure water |

(1) As shown in FIG. 1, the donepezil-loaded natural nanoparticle-drug composition is prepared according to the recombination ultrasonic method. 5 mg of the lipid-the phosphatidylcholine and 1 mg of the lipid-the cholesterol of a prescription dosage are weighed and added into a membrane-forming solvent (chloroform:methanol, 1:1) to form a lipid solution.

(2) 8 mg of the apolipoprotein apoA-I of a prescription dosage is weighed and dissolved in the ultrapure water to form an apolipoprotein solution.

(3) The drug-the donepezil of a prescription dosage is weighed and dissolved in the above lipid solution according to the principle of “like dissolves like”.

(4) The above lipid solution is subjected to rotary evaporation for 0.5 h at 37° C. to form a membrane, and overnight vacuum drying is carried out.

(5) The ultrapure water is added into the lipid membrane in step (4), and rotary evaporation is carried out at 37° C. for 5 min for hydration.

(6) After the hydration is finished, probe ultrasonic dispersion is carried out under an ice bath for 5 min to obtain drug-loaded nanoparticles.

(7) The apolipoprotein solution is added into the drug-loaded nanoparticles in step (6), and magnetically stirred at room temperature for incubation for 24 h. After the incubation is finished, a 0.22 μm filter membrane is utilized for filtration. Freeze-drying is carried out to obtain the donepezil-loaded natural nanoparticle-drug composition.

1.9 A recombination ultrasonic method for preparing a curcumin-loaded natural nanoparticle-drug composition

| | |
|---|---|
| Prescription composition: apoA-I | 6 mg |
| apoC | 2 mg |
| apoB100 | 2 mg |
| cholesterol | 5 mg |
| cholesterol ester | 1 mg |
| phosphatidylcholine | 2 mg |
| curcumin | 10 mg |
| hydration medium | ultrapure water |

(1) As shown in FIG. 1, the curcumin-loaded natural nanoparticle-drug composition is prepared according to the recombination ultrasonic method. 2 mg of the lipid-the phosphatidylcholine, 5 mg of the lipid-the cholesterol and 1 mg of the lipid-the cholesterol ester of a prescription dosage are weighed and added into a membrane-forming solvent (chloroform:methanol, 1:1) to form a lipid solution.

(2) 6 mg of the apolipoprotein apoA-I, 2 mg of the apoC and 2 mg of the apoB100 of a prescription dosage are weighed and dissolved in the ultrapure water to form an apolipoprotein solution.

(3) 10 mg of the drug-the curcumin of a prescription dosage is weighed and dissolved in the above lipid solution according to the principle of “like dissolves like”.

(4) The above lipid solution is subjected to rotary evaporation for 0.6 h at 37° C. to form a membrane, and overnight vacuum drying is carried out.

(5) The ultrapure water is added into the lipid membrane in step (4), and rotary evaporation is carried out at 37° C. for 10 min for hydration.

(6) After the hydration is finished, probe ultrasonic dispersion is carried out under an ice bath for 15 min to obtain drug-loaded nanoparticles.

(7) The apolipoprotein solution is added into the drug-loaded nanoparticles in step (6), and magnetically stirred at room temperature for incubation for 24 h. After the incubation is finished, a 0.22 μm filter membrane is utilized for filtration. Freeze-drying is carried out to obtain the curcumin-loaded natural nanoparticle-drug composition.

1.10 A recombination ultrasonic method for preparing a huperzine A-loaded natural nanoparticle-drug composition

| | | |
|---|---|---|
| Prescription composition: apoA-I | 7 | mg |
| apoA-II | 2 | mg |
| apoE | 1 | mg |
| triglyceride | 0.5 | mg |
| ceramide | 1 | mg |
| cephalin | 1 | mg |
| ganglioside | 1 | mg |
| huperzine A | 3 | mg |

| | |
|---|---|
| hydration medium | ultrapure water |

(1) As shown in FIG. 1, the huperzine A-loaded natural nanoparticle-drug composition is prepared according to the recombination ultrasonic method. 0.5 mg of the lipid-the triglyceride, 1 mg of the lipid-the ceramide, 1 mg of the lipid-the cephalin and 1 mg of the lipid-the ganglioside of a prescription dosage are weighed and added into a membrane-forming solvent (chloroform:methanol, 1:1) to form a lipid solution.

(2) 7 mg of the apolipoprotein apoA-I, 2 mg of the apoA-II and 1 mg of the apoE of a prescription dosage are weighed and dissolved in the ultrapure water to form an apolipoprotein solution.

(3) 3 mg of the drug-the huperzine A of a prescription dosage is weighed and dissolved in the above lipid solution according to the principle of “like dissolves like”.

(4) The above lipid solution is subjected to rotary evaporation for 0.5 h at 37° C. to form a membrane, and overnight vacuum drying is carried out.

(5) The ultrapure water is added into the lipid membrane in step (4), and rotary evaporation is carried out at 37° C. for 15 min for hydration.

(6) After the hydration is finished, probe ultrasonic dispersion is carried out under an ice bath for 20 min to obtain drug-loaded nanoparticles.

(7) The apolipoprotein solution is added into the drug-loaded nanoparticles in step (6), and magnetically stirred at room temperature for incubation for 24 h. After the incubation is finished, a 0.22 μm filter membrane is utilized for filtration. Freeze-drying is carried out to obtain the huperzine A-loaded natural nanoparticle-drug composition.

1.11 A recombination ultrasonic method for preparing an siRNA-loaded natural nanoparticle-drug composition

| | |
|---|---|
| Prescription composition: apoA-I | 5 mg |
| apoE | 2 mg |
| apoB | 3 mg |
| cholesterol | 1 mg |
| phosphatidylcholine | 4 mg |
| Chol-siRNA | 50 μg |
| hydration medium | ultrapure water |

(1) As shown in FIG. 1, the siRNA-loaded natural nanoparticle-drug composition is prepared according to the recombination ultrasonic method. 1 mg of the cholesterol and 4 mg of the phosphatidylcholine of a prescription dosage are weighed and added into a membrane-forming solvent (chloroform:methanol, 1:1) to form a lipid solution.

(2) 5 mg of the apolipoprotein apoA-I, 3 mg of the apoB and 2 mg of the apoE of a prescription dosage are weighed and dissolved in the ultrapure water to form an apolipoprotein solution.

(3) 50 μg of the drug-the Chol-siRNA of a prescription dosage is weighed and dissolved in the above apolipoprotein solution according to the principle of “like dissolves like”.

(4) The above lipid solution is subjected to rotary evaporation for 1 h at 37° C. to form a membrane, and overnight vacuum drying is carried out.

(5) The ultrapure water is added into the lipid membrane in step (4), and rotary evaporation is carried out at 37° C. for 25 min for hydration.

(6) After the hydration is finished, probe ultrasonic dispersion is carried out under an ice bath for 10 min to obtain nanoparticles.

(7) The apolipoprotein solution is added into the nanoparticles in step (6), and magnetically stirred at room temperature for incubation for 24 h. After the incubation is finished, a 0.22 μm filter membrane is utilized for filtration Freeze-drying is carried out to obtain the siRNA-loaded natural nanoparticle-drug composition.

1.12 A recombination ultrasonic method for preparing an NAP polypeptide-loaded natural nanoparticle-drug composition

| | |
|---|---|
| Prescription composition: apoA-I | 10 mg |
| phosphatidylcholine | 4 mg |
| cholesterol | 5 mg |
| NAP polypeptide | 100 μg |
| hydration medium | ultrapure water |

(1) As shown in FIG. 1, the NAP polypeptide-loaded natural nanoparticle-drug composition is prepared according to the recombination ultrasonic method. 5 mg of the cholesterol and 4 mg of the phosphatidylcholine of a prescription dosage are weighed and added into a membrane-forming solvent (chloroform:methanol, 1:1) to form a lipid solution.

(2) 10 mg of the apolipoprotein apoA-I of a prescription dosage is weighed and dissolved in the ultrapure water to form an apolipoprotein solution.

(3) 100 μg of the drug-the NAP polypeptide of a prescription dosage is weighed and dissolved in the above lipid solution according to the principle of “like dissolves like”.

(4) The above lipid solution is subjected to rotary evaporation for 1 h at 37° C. to form a membrane, and overnight vacuum drying is carried out.

(5) The ultrapure water is added into the lipid membrane in step (4), and rotary evaporation is carried out at 37° C. for 25 min for hydration.

(6) After the hydration is finished, probe ultrasonic dispersion is carried out under an ice bath for 10 min to obtain nanoparticles.

(7) The apolipoprotein solution is added into the nanoparticles in step (6), and magnetically stirred at room temperature for incubation for 24 h. After the incubation is finished, a 0.22 μm filter membrane is utilized for filtration Freeze-drying is carried out to obtain the NAP polypeptide-loaded natural nanoparticle-drug composition.

1.13 A recombination ultrasonic method for preparing a memantine-loaded natural nanoparticle-drug composition

| | |
|---|---|
| Prescription composition: apoE | 10 mg |
| phosphatidylcholine | 3 mg |
| memantine | 8 mg |
| hydration medium | ultrapure water |

(1) As shown in FIG. 1, the memantine-loaded natural nanoparticle-drug composition is prepared according to the recombination ultrasonic method. 3 mg of the phosphatidylcholine of a prescription dosage is weighed and added into a membrane-forming solvent (chloroform:methanol, 1:1) to form a lipid solution.

(2) 10 mg of the apolipoprotein apoE of a prescription dosage is weighed and dissolved in the ultrapure water to form an apolipoprotein solution.

(3) 8 mg of the drug-the memantine of a prescription dosage is weighed and dissolved in the above lipid solution according to the principle of “like dissolves like”.

(4) The above lipid solution is subjected to rotary evaporation for 0.5 h at 37° C. to form a membrane, and overnight vacuum drying is carried out.

(5) The ultrapure water is added into the lipid membrane in step (4), and rotary evaporation is carried out at 37° C. for 15 min for hydration.

(6) After the hydration is finished, probe ultrasonic dispersion is carried out under an ice bath for 15 min to obtain drug-loaded nanoparticles.

(7) The apolipoprotein solution is added into the drug-loaded nanoparticles in step (6), and magnetically stirred at room temperature for incubation for 24 h. After the incubation is finished, a 0.22 μm filter membrane is utilized for filtration Freeze-drying is carried out to obtain the memantine-loaded natural nanoparticle-drug composition.

1.14 A recombination ultrasonic method for preparing a methylene blue-loaded natural nanoparticle-drug composition

| | |
|---|---|
| Prescription composition: apoB | 10 mg |
| phosphatidylcholine | 4 mg |
| cholesterol | 2 mg |
| methylene blue | 14 mg |
| hydration medium | ultrapure water |

(1) As shown in FIG. 1, the methylene blue-loaded natural nanoparticle-drug composition is prepared according to the recombination ultrasonic method. 4 mg of the phosphatidylcholine and 2 mg of the cholesterol of a prescription dosage are weighed and added into a membrane-forming solvent (chloroform:methanol, 1:1) to form a lipid solution.

(2) 10 mg of the apolipoprotein apoB of a prescription dosage is weighed and dissolved in the ultrapure water to form an apolipoprotein solution.

(3) 14 mg of the drug-the methylene blue of a prescription dosage is weighed and dissolved in the above lipid solution according to the principle of “like dissolves like”.

(4) The above lipid solution is subjected to rotary evaporation for 1 h at 37° C. to form a membrane, and overnight vacuum drying is carried out.

(5) The ultrapure water is added into the lipid membrane in step (4), and rotary evaporation is carried out at 37° C. for 25 min for hydration.

(6) After the hydration is finished, probe ultrasonic dispersion is carried out under an ice bath for 25 min to obtain drug-loaded nanoparticles.

(7) The apolipoprotein solution is added into the nanoparticles in step (6), and magnetically stirred at room temperature for incubation for 24 h. After the incubation is finished, a 0.22 μm filter membrane is utilized for filtration Freeze-drying is carried out to obtain the methylene blue-loaded natural nanoparticle-drug composition.

### Embodiment 2: Study on the Properties of a Natural Nanoparticle-Drug Combination for Resisting Alzheimer's Disease

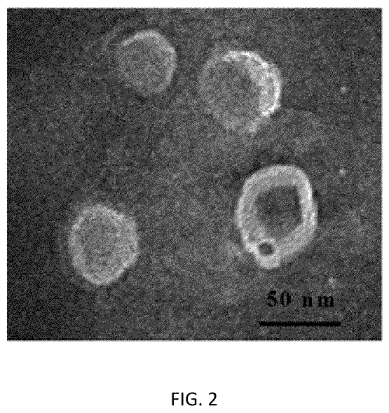

2.1 The donepezil-loaded natural nanoparticle-drug composition prepared according to Embodiment 1.1, particle size representation and morphological study: the particle size of the natural nanoparticle-drug composition is (65.8±4.45) nm as measured by a dynamic light scattering method, a spheroidal appearance (FIG. 2) is exhibited under a projection electron microscope, and the particle size is consistent with the particle size measured by a laser granulometer.

2.2 Study on an encapsulation rate and drug loading capacity of the donepezil-loaded natural nanoparticle-drug composition prepared according to Embodiment 1.1: 200 μL of the donepezil-loaded natural nanoparticle-drug composition is measured out in a 10 mL volumetric flask. Methanol is added for setting the volume to the scale. Water bath ultrasonic treatment is carried out for 30 min. Centrifuging (10000 rpm, 20 min) is carried out after demulsification. A supernatant is taken. The concentration of donepezil in the supernatant is measured by high performance liquid chromatography, and the encapsulation rate and the drug loading capacity are calculated according to the following formula: an encapsulation rate (%)=(mass of donepezil in a composition/total mass of added donepezil)×100%, and a drug loading capacity (%)=(mass of donepezil in a composition/total mass of a composition)×100%. The measured encapsulation rate of the donepezil-loaded natural nanoparticle-drug composition is 90% and the measured drug loading capacity is 5%.

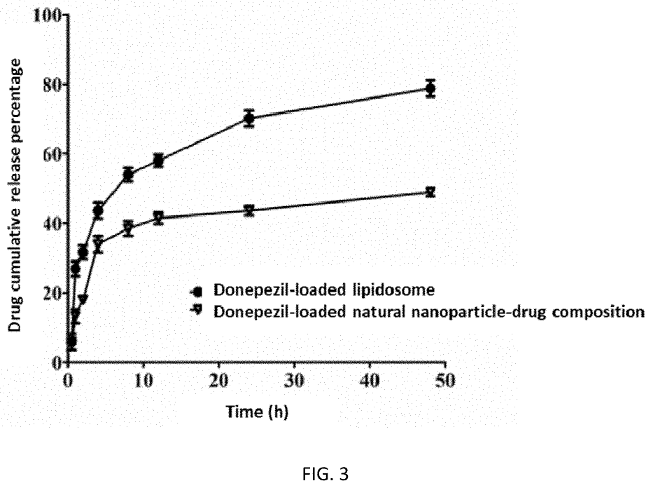

2.3 Study on in-vitro release of the donepezil-loaded natural nanoparticle-drug composition prepared according to Embodiment 1.1: 2 mL of the donepezil-loaded natural nanoparticle-drug composition is placed in a dialysis bag (molecular weight cut-off 3500), then put into 50 mL of a release medium (0.5% Tween-80, pH 7.4), and placed in a 37° C. constant temperature shaker. 1 mL of the release medium is taken out at a fixed point, and a blank release medium is supplemented to release drugs. As shown in FIG. 3, the donepezil-loaded natural nanoparticle-drug composition has only 0.5% of donepezil release within 1 h without significant drug burst release, and the cumulative release of the donepezil within 48 h is 51%, which is significantly lower than that of a lipidosome group. It can be seen that the donepezil-loaded natural nanoparticle-drug composition has a good drug slow-release effect.

### Embodiment 3: The Donepezil-Loaded Natural Nanoparticle-Drug Composition Prepared According to

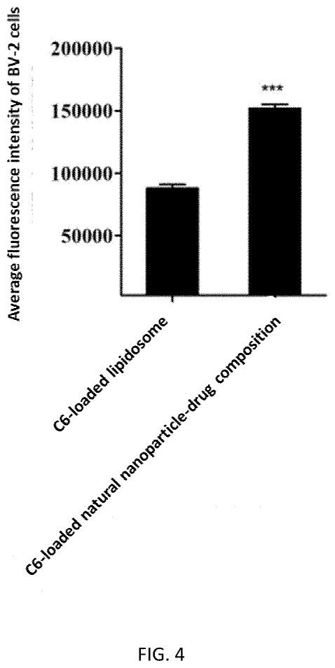

Embodiment 1.1: an in-vitro BBB model is constructed by Transwell co-culture. Human brain microvascular endothelial cells (hCMEC/D3) are inoculated in an upper Transwell chamber (6×10⁵ cells/cm²), mouse microglia cells (BV-2) are inoculated in a lower Transwell chamber (2×10⁵ cells/cm²), and cell transmembrane resistance is measured by a transmembrane resistance meter. On the seventh day of the co-culture, the transmembrane resistance is as high as 200 Ω/cm², which demonstrates that the Transwell BBB model has been successfully constructed. Drugs is substituted with fluorescent dye coumarin 6 (C6), and a C6-labeled natural nanoparticle-drug composition and a C6-labeled lipidosome are constructed respectively. 200 μL of the C6-labeled natural nanoparticle-drug composition and 200 μL of the C6-labeled lipidosome are added into the upper Transwell chamber successfully constructing the BBB model. After incubation for 4 h, pre-cooled PBS is added into the lower chamber for washing for 3 times, the cells are digested with trypsin and collected, and a small flow cytometer is utilized to perform quantitative statistics of fluorescence uptake of BV-2 cells. As shown in FIG. 4, the fluorescence intensity of cells co-incubated with the C6-labeled natural nanoparticle-drug composition is nearly twice that of the C6-labeled lipidosome, which demonstrates that the natural nanoparticle-drug composition may cross BBB endothelial cells to be taken by underlying neuroglia cells in a targeted mode, and has good brain targeting ability.

### Embodiment 4

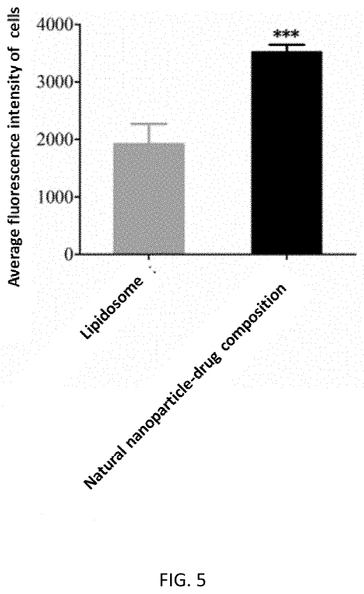

Study on Aβ targeted clearance capacity of the donepezil-loaded natural nanoparticle-drug composition prepared according to Embodiment 1.1: microglial cells BV-2 are inoculated in a 24-hole cell culture plate (2×10⁵ cells/hole), fluorescently labeled Aβ (FAM-Aβ) is added, and the natural nanoparticle-drug composition or a lipidosome is added for incubation for 4 h respectively. After the incubation is finished, a culture medium is sucked, the cells are washed three times with pre-cooled PBS, then the cells are digested with trypsin and collected, and a small flow cytometer is utilized to perform quantitative statistics of for fluorescence uptake of BV-2 cells. As shown in FIG. 5, the targeted uptake of Aβ by BV-2 cells co-incubated with the natural nanoparticle-drug composition is greatly improved over that of the lipidosome, which demonstrates that the natural nanoparticle-drug composition has a good Aβ targeted clearance capacity and AD treatment potential.

What is claimed is:

1. A natural nanoparticle-drug composition for treating Alzheimer's disease, wherein the natural nanoparticle-drug composition for treating Alzheimer's disease has brain targeting ability and amyloid protein targeting ability, and by a total mass of a prescription, content of natural nanoparticles accounts for 50-99% of the total mass of the prescription and content of drugs accounts for 1-50% of the total mass of the prescription.

2. The natural nanoparticle-drug composition for treating Alzheimer's disease of claim 1, wherein the natural nanoparticles are natural nanoparticles extracted from plasma components or expressed through genetic engineering and having an anti-Alzheimer's effect, and a particle size range of the natural nanoparticles is 20-200 nm.

3. The natural nanoparticle-drug composition for treating Alzheimer's disease of claim 2, wherein the natural nanoparticles are natural lipoprotein nanoparticles, and lipoprotein is essentially composed of apolipoprotein and lipid and is selected from one or more of high-density lipoprotein, low-density lipoprotein and very-low-density lipoprotein.

4. The natural nanoparticle-drug composition for treating Alzheimer's disease of claim 3, wherein the apolipoprotein is extracted from plasma or obtained through genetic engineering and is selected from one or more of apolipoprotein apoA-I, apoA-II, apoE, apoC, apoB and apolipoprotein mimetic peptide.

5. The natural nanoparticle-drug composition for treating Alzheimer's disease of claim 3, wherein the lipid is selected from one or more of cholesterol, cholesterol ester, phosphatidylcholine, triglyceride, ceramide, ganglioside and cephalin.

6. The natural nanoparticle-drug composition for treating Alzheimer's disease of claim 1, wherein Alzheimer's disease treatment drugs are selected from any one or derivatives of cholinesterase inhibitors, aspartic acid receptor antagonists, antioxidants, anti-inflammatory drugs, protein drugs, polypeptide drugs and gene drugs, and selected from one or more of donepezil, huperzine A, memantine, curcumin, methylene blue, NAP polypeptide or siRNA.

7. The natural nanoparticle-drug composition for treating Alzheimer's disease of claim 1, the natural nanoparticle-drug composition is prepared by the following steps:
(1) dispersing natural lipoprotein nanoparticles extracted from plasma or through genetic engineering into ultrapure water to form a natural nanoparticle suspension;
(2) dissolving drugs of a prescription dosage in a drug solvent, dropwise adding the natural nanoparticle suspension in step (1), and carrying out probe ultrasonic dispersion under an ice bath for 15-60 min; and
(3) after ultrasonic treatment is finished, when the drug solvent is an organic solvent, carrying out reduced pressure rotary evaporation for 5-20 min at 20-40° C. to remove the drug solvent; when the drug solvent is water, not carrying out rotary evaporation for removing the solvent; and then carrying out filtration through a 0.22 μm filter membrane, and freeze-drying to obtain the natural nanoparticle-drug composition.

8. The preparation method of the natural nanoparticle-drug composition for treating Alzheimer's disease of claim 7, wherein the drug solvent is that the solvent for a water-soluble drug is selected from one of ultrapure water, normal saline, a phosphate buffer solution and a Tris buffer solution, and the solvent for a fat-soluble drug is selected from any one of ethanol, methanol and acetone.

9. The natural nanoparticle-drug composition for treating Alzheimer's disease of claim 1, the natural nanoparticle-drug composition is prepared by a recombination ultrasonic method which comprises:
(1) weighing lipid of a prescription dosage, and adding the lipid into a membrane-forming solvent to form a lipid solution;
(2) weighing apolipoprotein of a prescription dosage, and dissolving the apolipoprotein in ultrapure water to form an apolipoprotein solution;
(3) weighing drugs of a prescription dosage, and dissolving the drugs in the above corresponding components according to the principle of “like dissolves like”: dissolving fat-soluble drugs in the lipid solution and water-soluble drugs in the apolipoprotein solution;
(4) carrying out rotary evaporation on the lipid solution at 20-40° C. for 0.5-2 h to form a membrane, and carrying out overnight vacuum drying;
(5) adding the ultrapure water into the lipid membrane in step (4) and carrying out rotary evaporation at 20-40° C. for 5-20 min for hydration;
(6) after the hydration is finished, carrying out probe ultrasonic dispersion under an ice bath for 5-20 min to obtain drug-loaded nanoparticles; and
(7) adding the apolipoprotein solution into the drug-loaded nanoparticles in step (6), and magnetically stirring at room temperature for incubation for 24-48 h; and after the incubation is finished, carrying out filtration through a 0.22 μm filter membrane, and freeze-drying to obtain the natural nanoparticle-drug composition.

10. A method for treating Alzheimer's disease with the natural nanoparticle-drug composition of claim 1 comprising a step of administrating the natural nanoparticle-drug composition to a subject needed for the treatment, wherein the natural nanoparticle-drug composition is diluted with a buffer solution, the buffer solution is selected from a group consisting of normal saline, a phosphate buffer solution, a 5% glucose solution, an HEPES buffer solution and a Tris buffer solution; the subject is administered via intravenous injection, or oral administration.
